(19) United States
(12) Patent Application Publication
(10) Pub. No.: US 2007/0255246 A1
(43) Pub. Date: Nov. 1, 2007

(54) Disposable absorbent articles with reinforced seams

(75) Inventor: Uwe Schneider, Wyoming, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/414,714

(22) Filed: Apr. 28, 2006

(51) Int. Cl. A61F 13/15

(52) U.S. Cl. 604/385.31; 604/385.3

(57) ABSTRACT

A disposable absorbent article has a chassis, a first side panel, a second side panel, and a reinforcement element. The chassis includes a topsheet, a backsheet attached to at least a portion of the topsheet, and an absorbent core disposed between the topsheet and the backsheet. The first side panel has an inner end and an outer end and extends outward from a first longitudinal edge in a first waist region. The second side panel extends outward from the first longitudinal edge in a second waist region. The first side panel is configured to join to the second side panel adjacent to the outer end of the first side panel. The reinforcement element is joined to the outer end and limited to the outer end of the first side panel.

### FIELD OF THE INVENTION

The present invention relates to disposable absorbent articles having seams which are reinforced. Specifically, the present invention relates to disposable diapers having reinforced fastening seams.

### BACKGROUND OF THE INVENTION

Disposable absorbent articles are widely used by infant and incontinent individuals. Some disposable absorbent articles are fastened about the wearer while donning the article on the wearer. In contrast, some disposable absorbent articles can be pre-fastened in a package such that donning the article on the wearer typically only involves pulling the disposable absorbent article onto the wearer.

The disposable absorbent article may comprise side panels which are capable of joining to one another via a re-fastenable fastening system. These re-fastenable fastening systems create attachments between the side panels which can be unattached and subsequently re-attached to one another.

Alternatively, the disposable absorbent article may comprise side panels which are joined together non-refastenably. For example, the side panels of the disposable absorbent article can be bonded together. In many cases these non-refastenable attachments cannot be unattached and effectively refastened after being unattached.

Whether the side panels are re-fastenably joined to one another or non-refastenably joined to one another, where the side panels overlap one another a seam is created.

In some instances, a removal tab can be implemented with at least one of the side panels which can facilitate the unattachment of the side panels. For example, where a non-refastenable fastening system is utilized, application of a force to the removal tab can break a frangible seam between side panels thereby unattaching the side panels. In another example, where a refastenable fastening system is utilized, application of a force to the removal tab can break the mechanical attachment between an engaging member and a receiving member of a re-fastenable fastening system thereby unattaching the side panels.

Where a removal tab is not present, wearers or caregivers often apply a force to a portion of the seam. For example, the wearer or caregiver may apply a force to an area of a side panel in the seam to break the frangible seam between the side panels thereby unattaching the side panels. As another example, the wearer or caregiver may apply a force to an area of a side panel in the seam to break a mechanical attachment between side panels.

In general, the removal tab and/or the side panels in the area of the seam should be strong enough to break the frangible seam or the mechanical attachment without itself tearing. However, in an effort to reduce costs, lighter materials and materials having a lower tensile strength can be utilized in a disposable absorbent article. Unfortunately, this may cause the removal tab and/or the side panels to lose tensile strength also.

Consequently, a need exists for a disposable absorbent article having reinforced seams. Also, a need exists for a disposable absorbent article having reinforced removal tabs.

### SUMMARY OF THE INVENTION

Disposable absorbent articles constructed in accordance with the present invention can have a reinforced fastening seam and/or a reinforced removal tab. In some embodiments, the disposable absorbent article for wearing about the lower torso of a wearer comprises a first waist region, a second waist region, a crotch region disposed between the first waist region and the second waist region; a first waist edge and a second waist edge; and a first longitudinal edge and a second longitudinal edge. The disposable absorbent article may further comprise a chassis, a first side panel, a second side panel, and a reinforcement element. The chassis comprises a topsheet, a backsheet attached to at least a portion of the topsheet, and an absorbent core disposed between the topsheet and the backsheet.

The first side panel extends outward from the first longitudinal edge in the first waist region and includes an inner end and an outer end. The second side panel extends outward from the first longitudinal edge in the second waist region, wherein the first side panel is configured to join to the second side panel adjacent to the outer end of the first side panel. The reinforcement element is joined to the outer end of the first side panel and is limited to the outer end of the first side panel.

In other embodiments, a disposable pant-like absorbent article for wearing about the lower torso of a wearer includes an outer cover having a front waist region, a back waist region, and a crotch region disposed between the front waist region and the back waist region, and a first backsheet layer which defines an outer surface of the disposable pant-like absorbent article.

The disposable pant-like absorbent article further comprises a first elastically extensible front side panel, a second elastically extensible front side panel, a first elastically extensible back side panel, and a second elastically extensible back side panel. The first elastically extensible front side panel and the second elastically extensible front side panel are each joined to a portion of the first backsheet layer and extend outward from the first backsheet layer. The first and the second the front side panels comprise a first inner end and a first outer end, wherein first front side panel comprises a first reinforcement element joined to the first outer end and limited to the first outer end of the first front side panel, and wherein the second front side panel comprises a first reinforcement element joined to the first outer end and limited to the first outer end of the second front side panel.

The first elastically extensible back side panel and the second elastically extensible back side panel are each joined to a portion of the first backsheet layer and extend outward from the first backsheet layer. The first and the second back side panels comprise a second inner end and a second outer end, wherein the first back side panel comprises a second reinforcement element joined to the second outer end and limited to the second outer end of the first back side panel, and wherein the second back side panel comprises a second reinforcement element joined to the second outer end and limited to the second outer end of the second back side panel. The first and the second elastically extensible front side panels are configured to join to the first and the second elastically extensible back side panels thereby creating a waist opening and a pair of leg openings.

The disposable pant-like absorbent article further comprises an absorbent assembly having a first longitudinal edge and a second longitudinal edge. The absorbent assembly comprises a topsheet, a second backsheet layer associated with the topsheet and an absorbent core disposed between the topsheet and the second backsheet layer. The second backsheet layer is disposed on the first backsheet layer, and the absorbent assembly is attached to the wearer-facing surface of the disposable pant-like absorbent article such that the first and second longitudinal edges are in a spaced apart relationship with each of the front and back side panels, thereby defining front longitudinally orientated non-elasticized portions and back longitudinally orientated non-elasticized portions therebetween.

The disposable pant-like absorbent article further comprises a first barrier leg cuff and a second barrier leg cuff. The first barrier cuff is attached to the first longitudinal edge of the absorbent assembly. The second barrier cuff is attached to the second longitudinal edge of the absorbent assembly.

### BRIEF DESCRIPTION OF THE DRAWINGS

FIG. 1A is a plan view showing a disposable absorbent article constructed in accordance with the present invention.

FIG. 1B is an isometric view showing the disposable absorbent article of FIG. 1A in a fastened state.

FIG. 2A is a cross sectional view showing a fastening seam of the disposable absorbent article of FIG. 1B through line 2A-2A.

FIG. 2B is a cross sectional view showing another embodiment of a fastening seam.

FIG. 3A is a cross sectional view showing reinforcement elements of the disposable absorbent article of FIG. 1A through line 3A-3A.

FIGS. 3B-3G are cross sectional views showing other embodiments for disposable absorbent articles constructed in accordance with the present invention.

FIG. 4 is a cross sectional view showing another embodiment for a disposable absorbent article constructed in accordance with the present invention.

FIGS. 5A-5C are cross sectional views showing several embodiments of reinforcement elements when comprising a portion of a backsheet.

FIGS. 6A-6C are cross sectional views showing several embodiments of reinforcement elements when comprising a portion of a cover layer.

FIG. 7 is a cross sectional view showing another embodiment of a disposable absorbent article constructed in accordance with the present invention.

FIG. 8 is a cross sectional view showing another embodiment of a disposable absorbent article constructed in accordance with the present invention.

### DETAILED DESCRIPTION OF THE INVENTION

### DEFINITIONS

As used herein, the terms “absorbent article” and “article” refer to a wearable device that absorbs and/or contains liquid and, more specifically, refers to a device that is placed against or in proximity to the body of the wearer to absorb and contain the various exudates discharged from the body. Suitable examples include diapers, training pants, refastenable pants, pull-on garments, adult incontinence products, and feminine care products such as sanitary napkins. Furthermore, the terms “absorbent article” and “article” include a “disposable absorbent article” which is intended to be discarded and not laundered or otherwise restored after no more than ten uses, preferably after no more than five uses, and most preferably after a single use (although certain components may be recycled, reused, or composted).

The terms “body-facing”, “wearer-facing”, “garment-facing”, and “outer-facing”, as used herein refer to the relative location of an element or a surface of an element or group of elements. “Body-facing” or “wearer-facing” implies the element or surface is nearer to the wearer during wear. “Garment-facing” or “outer-facing” implies the element or surface is more remote from the wearer during wear (i.e., element or surface is nearer to the wearer's garments that can be worn over the disposable absorbent article).

As used herein, the term “diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

As used herein “elastically extensible” refers to characteristics of extensible materials that have the ability to return to approximately their original dimensions after a force that extended the extensible material is removed. Herein, any material or element described as “extensible” may also be “elastically extensible” unless otherwise provided.

The term “fibrous substrate” refers to a web material which is made up a plurality of fibers. The fibers can be intermeshed with one another in random or uniform patterns. Some examples of substrates are wovens, nonwovens, or a combination thereof.

As used herein the term “joined” encompasses configurations whereby an element is directly secured to another element by affixing the element directly to the other element, and configurations whereby an element is indirectly secured to another element by affixing the element to an intermediate member(s) which in turn are affixed to the other element.

The term “longitudinal” is used herein to refer to a direction which is generally parallel to the longest edge of an element except where otherwise noted. In the context of some disposable absorbent articles, a “longitudinal” direction runs generally perpendicular from a waist edge to an opposing waist edge of the article and generally parallel to the maximum linear dimension of the article. Directions within ±45 degrees of the longitudinal direction are considered to be “longitudinal”.

The term “lateral” refers to a direction running generally perpendicular to and in the same plane as the “longitudinal” direction. In the context of disposable absorbent articles, a “lateral” direction runs from one longitudinal edge of the article to an opposing longitudinal edge of the article. Directions within ±45 degrees of the lateral direction are considered to be “lateral”.

The terms “pant”, “training pant”, “closed diaper”, “pre-fastened diaper”, and “pull-on diaper”, as used herein, refer to disposable garments having a waist opening and leg openings designed for infant or adult wearers. A pant can be configured such that the pant has a closed waist and leg openings prior to being donned on the wearer, or the pant can be configured such that the waist is closed and the leg openings formed while on the wearer. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened, rear waist fastened). Examples of suitable pants are disclosed in U.S. Pat. No. 5,246,433; U.S. Pat. No. 5,569,234; U.S. Pat. No. 6,120,487; U.S. Pat. No. 6,120,489; U.S. Pat. No. 4,940,464; U.S. Pat. No. 5,092,861; U.S. Pat. No. 5,897,545; U.S. Pat. No. 5,957,908; and U.S. Patent Publication No. 2003/0233082 A1.

As used herein the term “refastenable” refers to the attachment of two or more elements or portions of elements together via fastening elements. The fastening elements are joined in a manner in which they can be separated and re-attached without substantial degradation of fastener performance or damage to surrounding components of the article which would impair the article's continued use.

As used herein the term “non-refastenable” refers to the attachment of two or more elements or portions of elements together in a manner in which they cannot be separated and re-attached without substantial degradation to the elements, portions of elements, and/or surrounding components of the article.

The term “reinforcement element” is used herein to refer to a fibrous substrate which is disposed on a side panel. The “reinforcement element” does not include fastening elements or fastening components such as engagement hooks, loops, adhesive tapes, and the like.

### DESCRIPTION

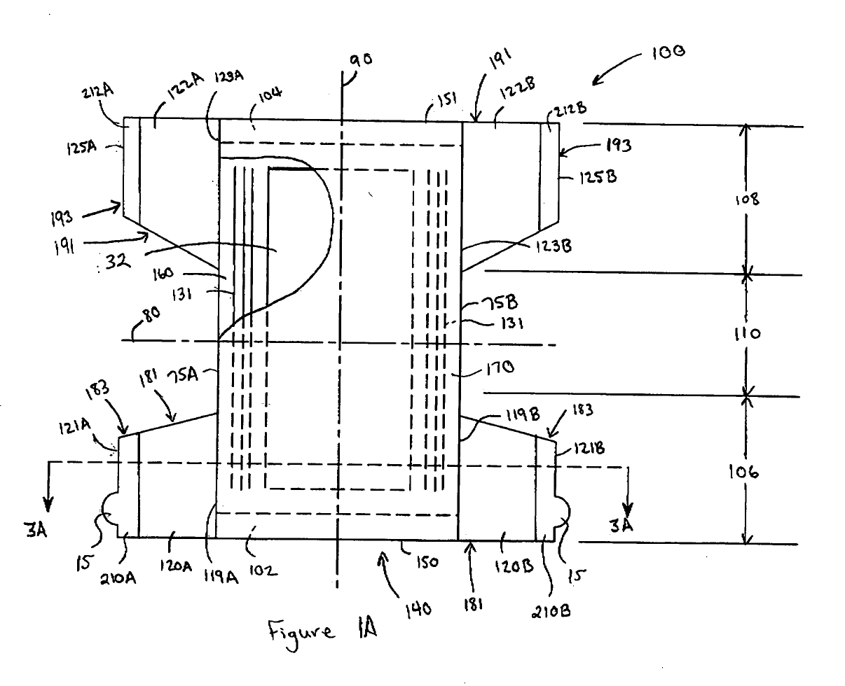

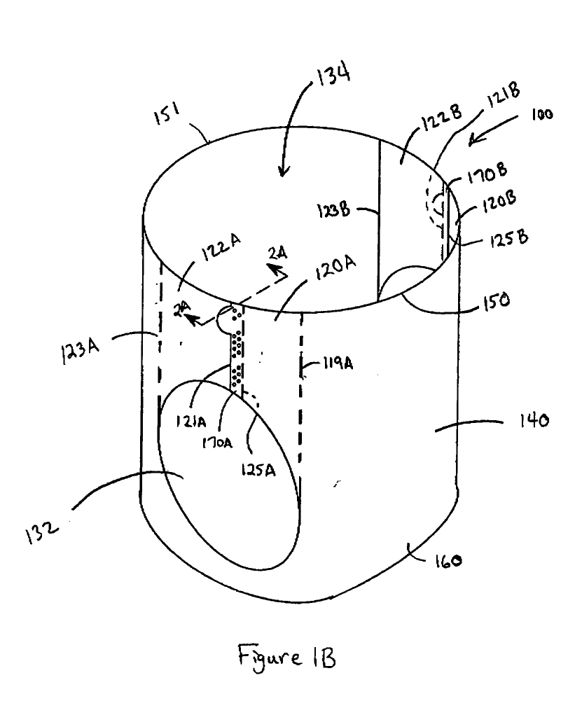

Disposable absorbent articles constructed in accordance with the present invention can provide a reinforced fastening seam and/or a reinforced removal tab. As shown in FIG. 1A, the portion of a disposable absorbent article 100 that faces a wearer is oriented towards the viewer. The disposable absorbent article 100 may comprise a first waist region 106, a second waist region 108, and a crotch region 110 disposed between the first waist region 106 and the second waist region 108. The disposable absorbent article 100 may further comprise a chassis 140. In some embodiments, the chassis 140 may comprise a topsheet 170, a backsheet 160, and an absorbent core 32. The absorbent core 32 can be positioned between at least a portion of the topsheet 170 and the backsheet 160.

The periphery of the chassis 140 can be defined by the longitudinal edges 75A and 75B; the first waist edge 150, and the second waist edge 151. The longitudinal edges 75A and 75B run generally parallel to a longitudinal centerline 90 of the disposable absorbent article 100. The first waist edge 150 and the second waist edge 151 run generally parallel to a lateral centerline 80 of the disposable absorbent article 100. The chassis 140 may further comprise elastic leg features 131 which can be disposed adjacent to the longitudinal edges 75A and 75B.

The disposable absorbent article 100, in some embodiments, may further comprise a first waist member 102 and a second waist member 104. The first waist member 102 and/or the second waist member 104 can be elastically extensible. As shown, in some embodiments, the first waist member 102 can be disposed adjacent the first waist edge 150. In some embodiments, the second waist member 104 can be disposed adjacent to the second waist edge 151. Generally, the first waist member 102 and/or the second waist member 104 can be under tension prior to attaching to the chassis 140. So, upon release of at least a portion of the tension applied to the first waist member 102 and/or the second waist member 104, a portion of the chassis 140 attached thereto can corrugate. This corrugation of the chassis 140 can allow the first waist member 102 and/or the second waist member 104 and the chassis 140 to expand and contract about the waist of a wearer, thereby providing more comfort and improved fit to a wearer.

A pair of first side panels 120A and 120B can extend outboard from the longitudinal edges 75A and 75B of the chassis 140 in the first waist region 106. A pair of second side panels 122A and 122B can extend outboard from longitudinal edges 75A and 75B of the chassis 140 in the second waist region 108. In some embodiments, the first side panels 120A and 120B can be elastically extensible. Similarly, in some embodiments, the second side panels 122A and 122B can be elastically extensible.

In some embodiments, the first and/or second side panels 120A, 120B, 122A, and 122B, can be formed of discrete separate elements affixed to the disposable absorbent article 100. In some embodiments, the first and/or second side panels 120A, 120B, 122A, and 122B, can be formed from a unitary piece of material that is neither divided nor discontinuous with an element of the disposable absorbent article 100. For example, in some embodiments, the side panels may comprise a portion of the topsheet 170. As another example, the side panels may comprise a portion of the backsheet 160. As yet another example, the side panels may comprise a portion of a barrier leg cuff (discussed hereafter).

The first side panel 120A comprises an inner end 119A and an outer end 121A. Similarly, the first side panel 120B comprises an inner end 119B and an outer end 121B. Additionally, each of the first side panels 120A and 120B comprise an inner zone 181 and an outer zone 183. The inner zones 181 are disposed adjacent to the inner ends 119A and 119B while the outer zones are disposed adjacent to the outer ends 121A and 121B.

The second side panel 122A also comprises an inner end 123A and an outer end 125A. The second side panel 122B comprises an inner end 123B and an outer end 125B. Similar to the first side panels 120A and 120B, each of the second side panels 122A and 122B comprise an inner zone 191 and an outer zone 193. The inner zones 191 are disposed adjacent to the inner ends 123A and 123B while the outer zones 193 are disposed adjacent to the outer ends 125A and 125B.

The first side panels 120A and 120B can be joined to the chassis 140 adjacent to their respective inner ends 119A and 119B. Similarly, the second side panels 121A and 121B can be joined to the chassis 140 adjacent to their respective inner ends 123A and 123B.

As shown, in some embodiments, the disposable absorbent article 100 may further comprise first reinforcement elements 210A and 210B. The first reinforcement elements 210A and 210B can be disposed adjacent to the outer ends 121A and 121B of the first side panels 120A and 120B, respectively. The first reinforcement elements 210A and 210B are limited to the outer zones 183 of the first side panels 120A and 120B, respectively.

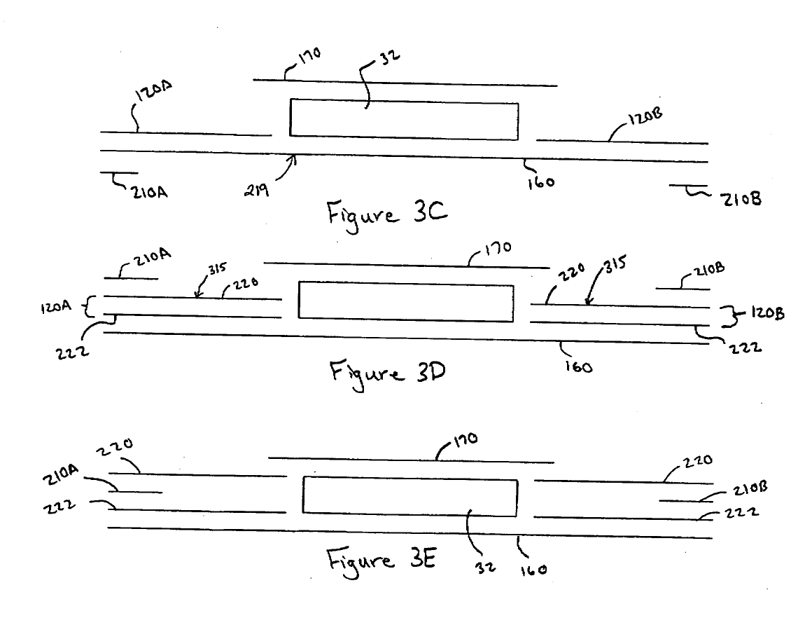

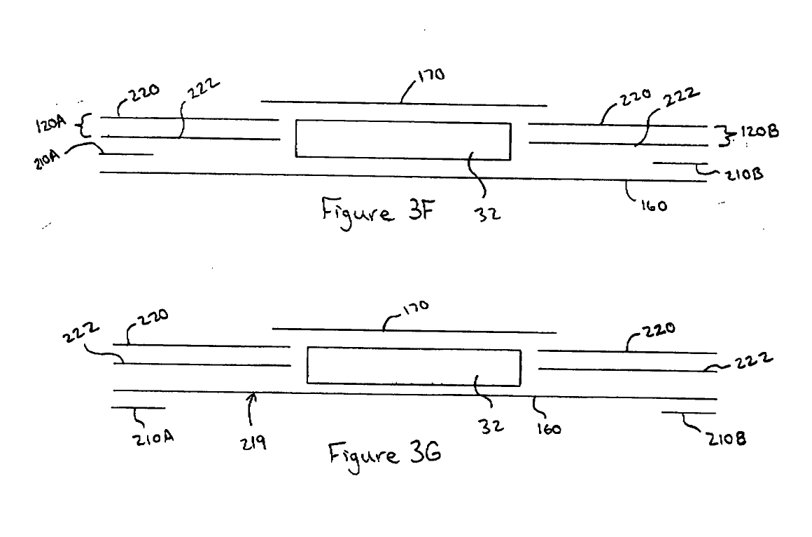

Additionally, in some embodiments, the disposable absorbent article 100 may further comprise second reinforcement elements 212A and 212B. The second reinforcement elements 212A and 212B can be disposed adjacent to the outer ends 125A and 125B of the second side panels 122A and 122B, respectively. The second reinforcement elements 212A and 212B are limited the outer zones 193 of the second side panels 122A and 122B, respectively. The inner ends; outer ends, inner zones, and outer zones, of the first side panels and the second side panels are discussed further with regard to FIG. 3A.

As shown in FIG. 1B, the first side panels 120A and 120B are configured to join to the second side panels 122A and 122B. For example, in some embodiments, the first side panel 120A can join to the second side panel 122A. Similarly, in some embodiments, the first side panel 120B can join to the second side panel 122B. The first side panel 120A and the second side panel 122A can be joined in a non-refastenable configuration. Alternatively, in some embodiments, the first side panel 120A and the second side panel 122A can be joined in a refastenable configuration.

As shown, the disposable absorbent article 100 is in a fastened state. The chassis 140 in combination with the first side panels 120A and 120B and the second side panels 122A and 122B can create a waist opening 134 and a pair of leg openings 132. The first side panels 120A and 120B and/or the second side panels 122A and 122B can form a portion of the leg openings 132 which would be disposed on an outer surface of a leg of a wearer. The crotch region 110 (shown in FIG. 1A) in conjunction with the first waist region 106 (shown in FIG. 1A) and the second waist region 108 (shown in FIG. 1A) can form a portion of the leg openings 132 which would be disposed on an inner surface of the leg of the wearer.

The joining of the first side panel 120A to the second side panel 122A can form the seam 170A. Similarly, the joining of the first side panel 120B to the second side panel 122B can form the seam 170B. The seams 170A and 170B as shown, in some embodiments, can be an overlap seam. In other embodiments, the seams 170A and the seam 170B can be a butt seam. In other embodiments, the seam 170A may be an overlap seam while the seam 170B is a butt seam or vice versa.

In some embodiments, the first side panel 120A can overlap the second side panel 122A by greater than or equal to about 2 mm. For example, in some embodiments, the first side panel 120A can overlap the second side panel 122A between about 2 mm to about 30 mm or any individual number within the range. In some embodiments, the overlap can be between about 11 mm to about 22 mm. In some embodiments, the first side panel 120A can overlap the second side panel 122A by a first distance adjacent the waist opening 134 and can overlap the second side panel 122A by a second distance adjacent the leg opening 132. In some embodiments, the first distance is equal to the second distance. In some embodiments, the first distance is greater than the second distance. In some embodiments, the second distance is greater than the first distance. In some embodiments, the first side panel 120B and the second side panel 122B can overlap each other as described above.

Although the first side panel 120A is shown on the garment-facing surface of the seam 170A, in some embodiments, the second side panel 122A may form a portion of the garment-facing surface of the seam 170A. The side panels 120B and 122B can be similarly configured.

As shown, in some embodiments, the first side panel 120A can join the second side panel 122A adjacent to the outer end 121A of the first side panel 120A. The first side panel 120A can be joined to the second side panel 122A by any suitable means known in the art. For example, the first side panel 120A may be compression bonded to the second side panel 122A. In yet another example, the first side panel 120A may be adhesively joined to the second side panel 122A. In yet other examples, the first side panel 120A may be ultrasonically bonded to the second side panel 122A. In yet other examples, the first side panel 120A may be bonded via hot air to the second side panel 122A. Hot air bonding is described in U.S. Pat. No. 6,248,195. In yet other examples, the first side panel 120A may comprise a first mechanical fastening element while the second side panel 122A comprises a second mechanical fastening element. The first mechanical fastening element can configured to engage the second mechanical fastening element thereby forming a mechanical bond between the first side panel 120A and the second side panel 122A. Other examples of suitable means for joining the first side panels to the second side panels include cohesives, adhesives, mechanical fasteners, the like, or combinations thereof.

As shown in FIG. 1A, the first side panels 120A and 120B may further comprise removal tabs 15. The removal tabs 15 may facilitate the attachment of the first side panels 120A and 120B to the second side panels 122A and 122B, respectively. Also, the removal tab 15 may facilitate the unattachment of the first side panels 120A and 120B from the second side panels 122A and 122B, respectively.

An advantage of the present invention is that the seams 170A (shown in FIG. 1B) and/or 170B (shown in FIG. 1B) can be reinforced by the reinforcement elements 210A, 210B, 212A, and/or 212B. Additionally, where the first side panel 120A and/or the first side panel 120B comprise the removal tab 15, the removal tab 15 can similarly be reinforced by the reinforcement elements 210A and/or 210B.

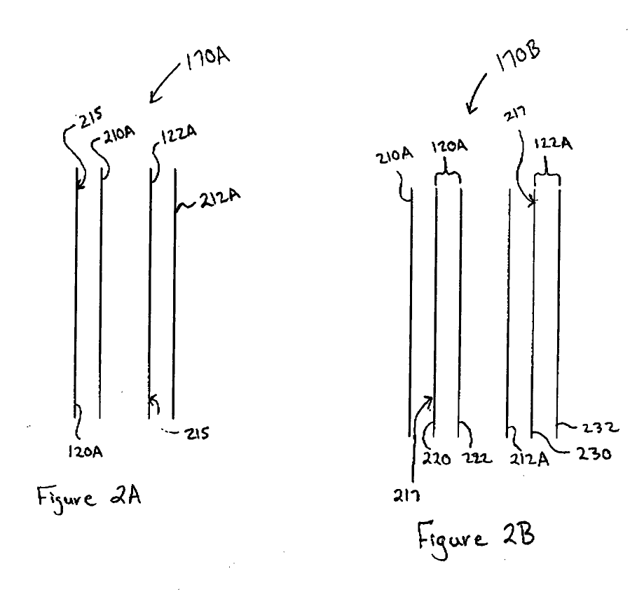

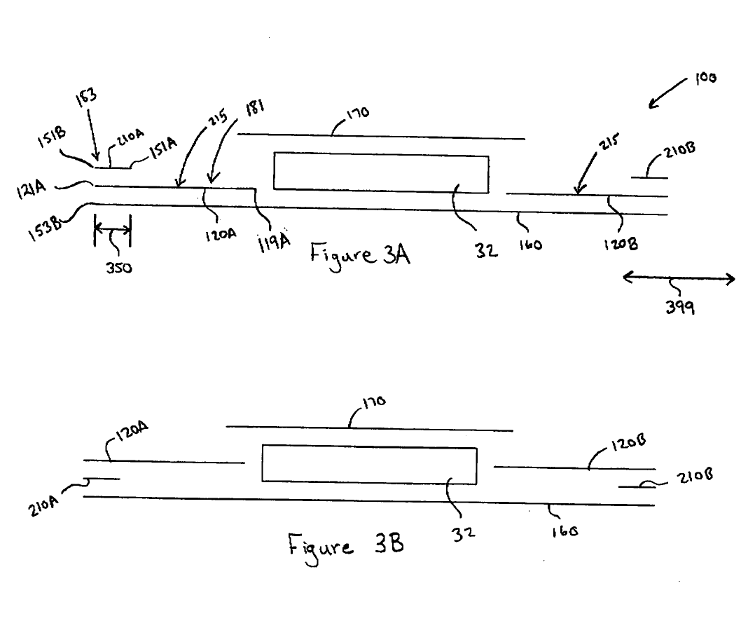

As shown in FIG. 2A, and as stated previously, the seam 170A may comprise an overlap seam, in some embodiments. For example, as shown, a portion of the first side panel 120A may overlap a portion of the second side panel 122A. Additionally, as shown, in some embodiments, the first reinforcement element 210A can be disposed on a wearer-facing surface 215 of the first side panel 120A. In some embodiments, the second reinforcement element 212A can be disposed on a wearer-facing surface 215 of the second side panel 122A.

As shown in FIG. 2B, in some embodiments, the first side panel 120A may comprise an elastomeric element 222 and a cover layer 220. Similarly, in some embodiments, the second side panel 122A may comprise an elastomeric element 232 and a cover layer 230. In some embodiments, the first reinforcement element 210A can be disposed on an outer-facing surface 217 of the first side panel 120A. In some embodiments, as shown, the second reinforcement element 212A can be disposed on an outer-facing surface 217 of the second side panel 122A.

The reinforcement elements of the present invention may comprise portion of other elements. For example, in some embodiments, the reinforcement elements may comprise a portion of the backsheet 160. In other embodiments, the reinforcement elements may comprise a portion of the cover layers 230 and/or 220. In some embodiments, the reinforcement elements may comprise discrete separate elements which are joined to the disposable absorbent article.

As shown in FIG. 3A, in some embodiments, the reinforcement elements 210A and/or 210B can be disposed on the wearer-facing surface 215 of the first side panel 120A and/or the first side panel 120B, respectively. Additionally, in some embodiments, the reinforcement elements 210A and/or 210B can be disposed between the topsheet 170 and the backsheet 160.

As shown, the reinforcement element 210A can have a reinforcement element width 350 which is generally parallel to a lateral direction 399. The reinforcement element width 350 can be the maximum linear distance between an inwardmost point 151A and an outwardmost point 151B of the reinforcement element 210A. In some embodiments, the width 350 can be between about 5 mm to about 40 mm or any individual number within the range. In other embodiments, the width 350 can be between about 10 mm to about 30 mm. In other embodiments, the width 350 can be between about 15 mm and about 20 mm.

Additionally, in some embodiments, the reinforcement element 210A can define a portion of the outer end 121A of the first side panel 120A. In some embodiments, the reinforcement element 210A can be disposed inward from the outer end 121 A of the first side panel 120A. As stated previously, the reinforcement element 210A can define the outer zone 183.

The outer zone 183 is defined by the inwardmost point 151A of the first reinforcement element 210A and the outer end 121A of the first side panel 120A. In some embodiments, the reinforcement element 210A is coextensive with the outer zone 183.

As shown, in some embodiments, the inner zone 181 can extend from the inner end 119A to the inwardmost point 151A of the reinforcement element 210A. Additionally, the inwardmost point 151A of the reinforcement element 210A is disposed outboard of inner end 119A of the first side panel 120A.

As shown in FIG. 3B, in some embodiments, the reinforcement elements 210A and/or 210B can be disposed between the first side panel 120A and the backsheet 160 and the first side panel 120B and the backsheet 160, respectively. As shown in FIG. 3C, in some embodiments, the reinforcement elements 210A and/or 210B can be disposed on a garment-facing surface 219 of the backsheet 160.

As shown in FIG. 3D, in some embodiments, as discussed previously the first side panel 120A and/or 120B may comprise the cover layer 220 and the elastomeric element 222. As shown, in some embodiments, the reinforcement elements 210A and/or 210B can be disposed on a wearer-facing surface 315 of the cover layer 220 of the first side panel 120A and the first side panel 120B, respectively.

As shown in FIG. 3E, in some embodiments, reinforcement elements 210A and/or 210B can be disposed between the cover layer 220 and the elastomeric element 222 of the first side panel 120A and the first side panel 120B, respectively. As shown in FIG. 3F, in some embodiments, reinforcement elements 210A and/or 210B can be disposed between the elastomeric element 222 and the backsheet 160 of the first side panel 120A and the first side panel 120B, respectively. As shown in FIG. 3G, in some embodiments, the reinforcement elements 210A and/or 210B can be disposed on an outer-facing surface 219 of the backsheet 160.

The second side panel 122A (shown in FIGS. 1A and 1B) and/or the second side panel 122B (shown in FIGS. 1A and 1B) can be configured in a similar manner to the first side panels 120A and 120B. As discussed previously, the second side panels 122A and 122B may include reinforcement elements. For example, the reinforcement element 212A (shown in FIGS. 1A, 2A and 2B) may correspond to the second side panel 122A and can be configured similarly to the reinforcement elements 210A and 210B as described above. Additionally, the reinforcement element 212B (shown in FIG. 1A) can be configured in a similar manner to the reinforcement elements 210A and 210B described heretofore.

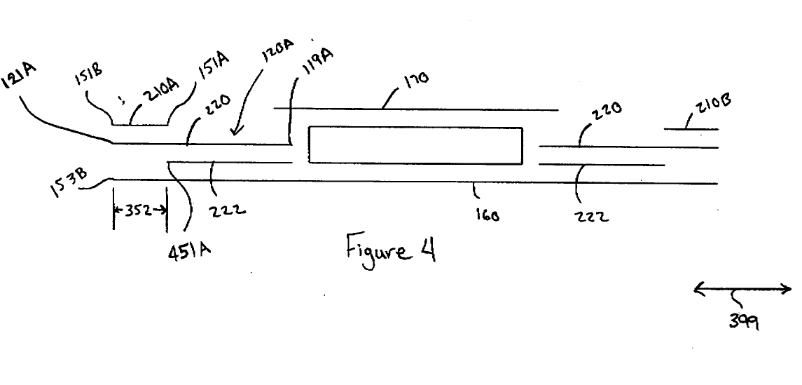

In other embodiments, as shown in FIG. 4, the elastomeric element 222 can be configured such that the elastomeric element 222 and the cover layer 220 are non-coextensive. For example, an outermost end 411B of the elastomeric element 222 can be disposed inboard of the spaced apart from an outermost end 153B of the backsheet 160 and/or the outer end 121A of the side panel by an inelastic gap 352. The inelastic gap 352 can be generally parallel to the lateral direction 399 and may extend longitudinally adjacent the outer end 121A. The inelastic gap 352 can be any suitable length. In some embodiments, the inelastic gap 352 can be equal to the width 350 (shown in FIG. 3A) of the reinforcement element 210A. In other embodiments, the elastomeric element 222 can be configured such that no portion of the elastomeric element 222 overlaps the reinforcement element 210A. In some embodiments, the elastomeric element 222 can be configured such that the the elastomeric element 222 extends to the outer end 121A of the side panel.

In some embodiments, the inelastic gap 352 can be between about 5 mm to about 30 mm or any individual number within the range. In some embodiments, the inelastic gap 352 can be between about 10 mm to about 20 mm. The first side panel 120B, the second side panel 122A and/or the second side panel 122B may be configured similarly.

When the elastomeric elements 222 comprise high quality elastomeric materials, the inclusion of the inelastic gap 352 can equate to cost savings. For example, utilization of the inelastic gap 352 can reduce the amount of elastomeric material utilized in the side panels while allowing the disposable absorbent article to provide comparable fit and stretch to a wearer as compared to prior disposable absorbent articles.

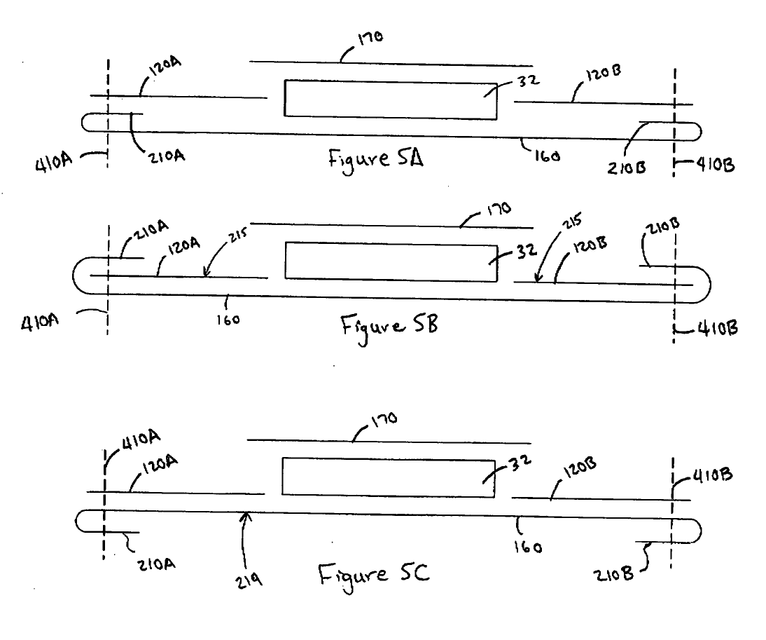

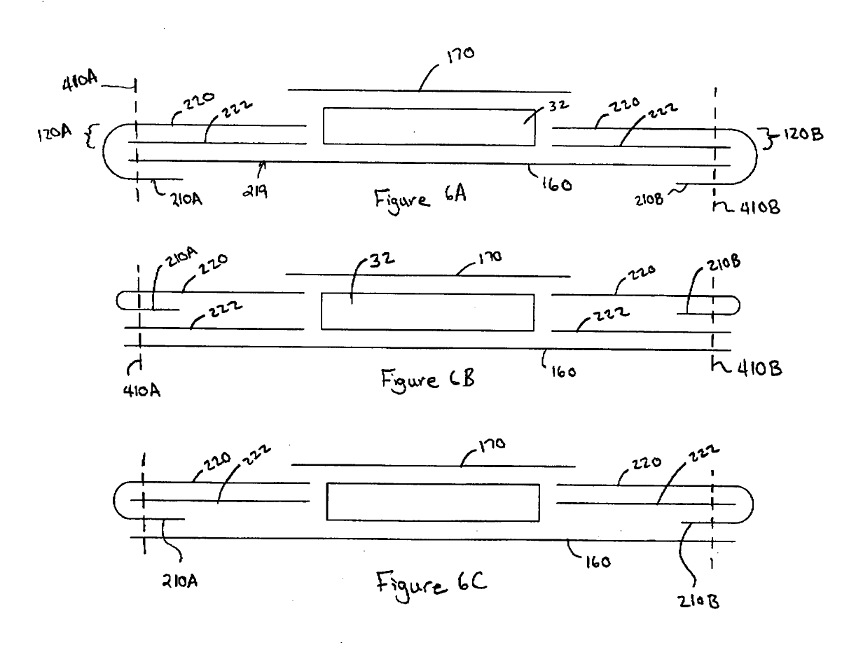

The reinforcement elements described heretofore can be implemented in a disposable absorbent article in a number of different ways. For example, the reinforcement elements can be placed on the disposable absorbent article as discrete pieces of material during the processing of the disposable absorbent article. In other embodiments, the reinforcement elements may be constructed utilizing portions of other elements, e.g. backsheet, cover layer, and/or topsheet. Examples of suitable embodiments are discussed with regard to FIGS. 5A-5C and 6A-6C.

As shown in FIGS. 5A-5C, in some embodiments, the backsheet 160 can be folded during the manufacturing process of an article such that the reinforcement elements 210A and 210B comprise a portion of the backsheet 160. For example, as shown in FIG. 5A, the backsheet 160 can be folded such that the reinforcement elements 210A and/or 210B are disposed between the first side panel 120A and the backsheet 160 and/or the first side panel 120B and the backsheet 160, respectively. In another example, as shown in FIG. 5B, the backsheet can be folded such that the reinforcement elements 210A and/or 210B are disposed on the wearer-facing surface 215 of the first side panels 120A and/or 120B, respectively. In yet another example, as shown in FIG. 5C, the backsheet 160 can be folded such that the reinforcement elements 210A and/or 21 0B are disposed on the outer-facing surface 219 of the backsheet 160.

One particular advantage to the embodiments shown in FIGS. 5A-5C is that the backsheet can be made from a lower basis weight nonwoven than in embodiments where the backsheet is not folded. For example, in many processes, the disposable absorbent articles, as they are processed, are cut along a first trim line 410A and a second trim line 410B. The trimming process can provide a disposable absorbent article with an hourglass contour.

In instances where the backsheet is not folded, the basis weight of the substrate used for the backsheet can be, for example, about 27 gsm. In these applications, where the backsheet is not folded, lighter basis weight substrates may break during the trimming process thereby causing a defect in the process and causing the processing line to stop. However, in embodiments, where the backsheet is folded, e.g. FIGS. 5A-5C, the trimmed portions of the disposable absorbent article are less likely to break because of the folded portions of the backsheet. Consequently, lighter basis weights can be utilized for the backsheet without increasing the likelihood of defects during the trim process. Suitable basis weights for the backsheet are discussed hereafter.

As shown in FIGS. 6A-6C, the cover layers 220 of the first side panels 120A and 120B can be folded such that the reinforcement elements 210A and/or 210B comprise a portion of the cover layers 220. For example, as shown in FIG. 6A, the cover layers 220 can be folded such that the reinforcement elements 210A and/or 210B are disposed on the outer-facing surface 219 of the backsheet 160. In another example, as shown in FIG. 6B, the cover layers 220 can be folded such that the reinforcement elements 210A and/or 210B are disposed between the cover layers 220 and the elastomeric elements 222. In yet another example, as shown in FIG. 6C, the cover layers 220 can be folded such that the reinforcement elements 210A and/or 210B are disposed between the elastomeric elements 222 and the backsheet 160.

The reinforcement elements 210A and 210B discussed heretofore can be configured similarly. However, the reinforcement elements 210A and 210B are not required to be configured similarly. For example, any combination of the above embodiments can be utilized within a disposable absorbent article. Additionally, the reinforcement elements 212A (shown in FIGS. 1A, 2A, and 2B) and/or 212B (shown in FIG. 1A) can be configured as described heretofore. Embodiments where the backsheet 160 and/or the cover layer 220 are folded and not trimmed are contemplated.

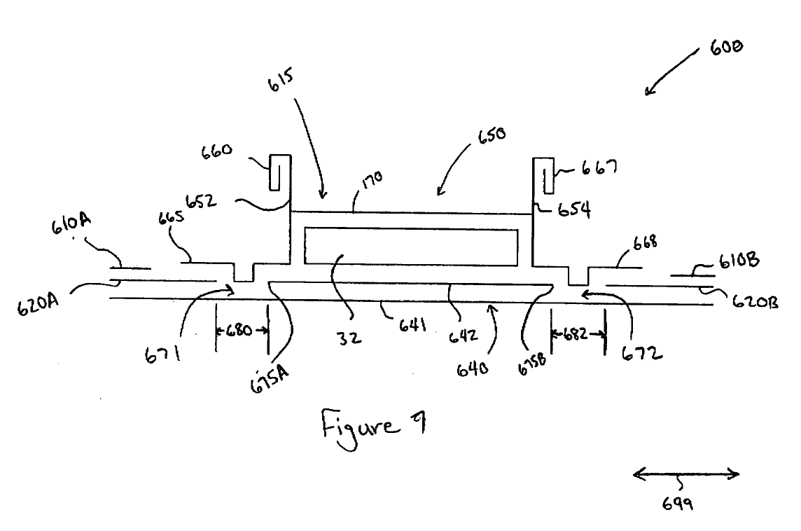

FIG. 7 is an exaggerated cross sectional view of a disposable absorbent article 600 which is constructed in accordance with the present invention. The disposable absorbent article 600 may comprise an outer cover 640 which includes a first backsheet layer 641, a front waist region, a back waist region, and a crotch region disposed therebetween. The disposable absorbent article 600 may further comprise an absorbent assembly 650. The absorbent assembly 650 may include a first longitudinal edge 675A and a second longitudinal edge 675B. The absorbent assembly 650 may also include the topsheet 170, a second backsheet layer 642, and the absorbent core 32. The absorbent assembly 650 can be joined to the first backsheet layer 641 such that the absorbent assembly 650 forms a portion of a wearer-facing surface 615 of the disposable absorbent article 600. Additionally, in some embodiments, the absorbent assembly 650 can be joined to the outer cover 640 such that the first longitudinal edge 675A is spaced apart from a first side panel 620A by a first gap 680 and the second longitudinal edge 675B is spaced apart from a first side panel 620B by a second gap 682.

In some embodiments, the first side panel 620A can be disposed on the wearer facing surface of the first backsheet layer 641. In some embodiments, the side panel 620B can be disposed on the wearer facing surface of the first backsheet layer 641. The side panel 620A and the first side panel 620B can be configured similarly to the side panels discussed herein.

The disposable absorbent article 600 may further comprise at least two barrier leg cuffs 652 and 654 attached to the topsheet 170 adjacent to the longitudinal edges 675A and 675B of the absorbent assembly 650. The barrier leg cuffs 652 and 654 can also be attached to the first backsheet layer 641.

The barrier leg cuff 652 comprises a distal region 660 and a proximal region 665 while the barrier leg cuff 654 comprises a distal region 667 and a proximal region 668. The proximal region 665 of the barrier leg cuff 652 can be joined to the first side panel 620A and the second backsheet layer 642 in an overlapping manner. Similarly, the proximal region 668 of the barrier leg cuff 654 can be joined to first side panel 620B and the second backsheet layer 642 in an overlapping manner. The distal region 660 of the barrier leg cuff 652 can be joined to the topsheet 170 and the distal region 667 of the barrier leg cuff 654 can be joined to the topsheet 170.

As shown, in some embodiments, the barrier leg cuffs 652 and 654 can be joined to the first backsheet layer 641 between the first side panels 620A and 620B, respectively, and the second backsheet layer 642. The attachment of the barrier leg cuffs 652 and 654 to the first backsheet layer 641 can define the first gap 680 and the second gap 682. The first gap 680 can extend longitudinally for a length of the first side panel 620A. Similarly, the second gap 682 can extend longitudinally for a length of the first side panel 620B. The first gap 680 can define a width of a first non-elastic region 671 of the disposable absorbent article 600 disposed between the first side panel 620A and the second backsheet layer 642, and the second gap 682 can define a width of a second non-elastic region 672 of the disposable absorbent article 600 disposed between the first side panel 620B and the second backsheet layer 642.

The inclusion of the non-elastic regions 671 and 672 can offer many advantages over previous disposable pant-type garments, such as pull-on diapers. For example, the inclusion of the non-elastic regions 671 and 672, when the first side panel 620A and the first side panel 620B comprise high quality elastomeric materials, can mean a cost savings. This savings in elastomeric materials can translate to a significant cost savings for disposable pull-on garments, which are produced in the hundreds of thousands, if not more, daily. That is, the amount of elastomeric material used in the first side panel 620A, the first side panel 620B, and the second side panels of the disposable absorbent article is minimized, while the disposable absorbent article still provides comparable, if not superior, fit and stretch to prior disposable pant-type garments.

The first and second gaps 680 and 682 may be of any suitable width in a lateral direction 699. For example, in one embodiment, the first and the second gaps 680 and 682 may range in width from about 0.5 mm to about 26 mm or any individual number within the range. In another embodiment, the first and the second gaps 680 and 682 may range in width from about 0.5 mm to about 20 mm. In yet another embodiment, the first and the second gaps 680 and 682 may range in width from about 1 mm to about 15 mm. The first gap 680 and the second gap 682, along with the non-elastic regions 671 and 672, are discussed in U.S. Patent Application Publication No. 2004/0225273 A1.

The barrier leg cuffs 652 and 654 can provide improved containment of liquids and other body exudates. In addition, these barrier leg cuffs 652 and 654 may include several different embodiments for reducing the leakage of body exudates in the leg regions. Illustrative examples of suitable barrier leg cuffs for use in the present invention may be found in U.S. Pat. No. 3,860,003, U.S. Pat. No. 4,909,803, U.S. Pat. No. 4,695,278, U.S. Pat. No. 4,795,454, U.S. Pat. No. 4,704,115, and U.S. Pat. No. 4,808,178.

In addition, the barrier leg cuffs 652 and 654 may be attached to the disposable absorbent article 100C by any suitable attachment means or any suitable combination of attachment means known in the art. Some examples of suitable attachment means include, but are not limited to, adhesive bonds, heat bonds, pressure bonds, ultrasonic bonds, and dynamic mechanical bonds. Additionally, in some embodiments, it may be desirable to treat all or a portion of the leg cuffs with a lotion or a fecal modification agent which either increases or decreases the Hardness of fecal material which it encounters. Lotions as well as fecal modification agents are discussed in U.S. Patent Publication No. 2004/0039362A1 and U.S. Pat. No. 6,639,119.

The disposable absorbent article 600 may further comprise reinforcement elements 610A and 610B. The reinforcement elements 610A and 610B can be disposed on the wearer-facing surface 615 of the disposable absorbent article 600, in some embodiments. The reinforcement elements 610A and 610B can be configured and created similarly to the reinforcement elements described heretofore.

A disposable absorbent article of the present invention comprises many different members/elements, e.g. reinforcement elements, a topsheet, a backsheet, an absorbent core, etc. for which a wide assortment of materials can be used. For example, any suitable material can be used for the reinforcement element and/or cover layer. The reinforcement element and/or cover layer can be compliant and soft. Additionally, because the reinforcement element and/or cover layer can contact the skin of the wearer in some embodiments, the reinforcement element and/or cover layer can also be non-irritating to the skin of the wearer. Exemplary reinforcement elements and/or cover layers can comprise a nonwoven.

Suitable examples of nonwovens for use as a reinforcement elements, cover layers, and/or a portion of a backsheet, may comprise fibers made of polypropylene, polyethylene, polyester, nylon, cellulose, polyamide, or combinations of such materials. Fibers of one material or fibers of different materials or material combinations may be used in the first and/or second nonwoven. Exemplary nonwoven materials include spunbond, spunbond meltblown spunbond (SMS), spunbond meltblown meltblown spunbond (SMMS), carded and the like. Particularly acceptable nonwovens include high elongation carded (HEC) nonwovens and deep activation polypropylene (DAPP) nonwovens. Any process known in the art may be used to make the nonwovens.

The nonwoven may comprise fibers that are bonded internally, including fibers that are needle punched, hydro entangled, spun bonded, thermally bonded, bonded by various types of chemical bonding such as latex bonding, powder bonding, and the like. In certain embodiments, the basis weight of the nonwoven can be in the range of about 10 gsm to about 40 gsm or any individual number within the range.

The fibers may be of any suitable size. In some embodiments, the fiber may have a denier ranging from about 1 to about 10 or any individual number within the range. In some embodiments, the denier of the fibers can range from about 1 to about 8. In other embodiments, the denier of the fibers can range from about 1 to about 5.

In some embodiments, the reinforcement elements may comprise a bonding agent. For example, the reinforcement elements may comprise an adhesive. Any suitable adhesive can be used in the present invention. For example, the adhesive may comprise styrene-olefin-styrene triblock copolymers such as styrene-isoprene-styrene, styrene-butadiene-styrene, or combinations thereof.

As another example, the reinforcement elements may comprise a polymer. In some embodiments, the reinforcement elements may comprise a low melt high modulus polymer. Any suitable polymer known in the art can be utilized. Some examples of suitable polymers include a high modulus hot melt polymer or may include a molten polymer. Any suitable molten polymer can be used. Some examples of molten polymers include polyethylene, polypropylene, the like, or any suitable combinations thereof.

In some embodiments, the bonding agent can be applied at basis weights ranging from about 15 gsm to about 100 gsm or any individual number within the range. In some embodiments, the bonding agent can be applied at a basis weight ranging from about 30 gsm to about 80 gsm. In some embodiments, the bonding agent can be applied at a basis weight ranging from about 50 gsm to about 60 gsm.

The elastomeric element of the present invention may include elastic strands and/or elastic films. Any suitable elastic film known in the art can be used. Suitable elastic films may comprise polypropylene, polyethylene, polyolefins, styrene-isoprene-styrene, styrene-butadiene-styrene, or combinations thereof. The basis weight of the elastic films can range from about 10 gsm to about 100 gsm or any individual number within the range.

Examples of suitable elastic strands can be made of a resilient elastic thermoplastic material. The elastic strands may be made from liquid elastic that is extruded through a die to achieve the desired strand elastic diameter and/or shape. The shape of the extruded elastic strands is not limited. For example, typical elastic strands have a circular cross sectional shape, but sometimes the elastic strands may have different shapes, such as a trilobal shape, or a flat (i.e., “ribbon” like) shape. Suitable elastic strand shapes include rectangles, circles, ellipses, diamonds, triangles, parallelograms, trapezoids, wedges or other sections of circles or ellipses, other polygons, or other irregular enclosed shapes. Furthermore, the thickness or diameter of the elastic strands may vary in order to accommodate a particular application. Typically, the thickness of elastic strands may be in the range of about 0.02 mm to about 1 mm and the basis weight is in the range of about 20 g/m² to about 300 g/m² or any individual number within the range.

The elastic strands may be applied to a substrate by any suitable process. For example, in some embodiments, the elastic strands may be applied to a substrate via extruding and/or printing. Examples of suitable apparatuses for applying elastic strands onto a substrate or extruding a elastic strands onto a substrate are described in U.S. Application Publication No. 2004/0238105 A1; U.S. Application Publication No. 2005/0241773; U.S. Application Publication No. 2005/0178494; U.S. Application Publication No. 2005/0241575; and U.S. Application Publication No. 2005/0241574.

Examples of suitable apparatuses and methods for printing elastic elements in any orientation are described in U.S. Application Publication No. 2004/0181200; and in U.S. Application Publication No. 2004/0193133. For the printing of elastic strands, the individual elastic strands may be configured as lines or strands generally having widths less than about 2 mm and typically less than about 1 mm. Linear elastic strands may be configured as bands generally having widths between about 2 mm and about 20 mm and aspect ratios ranging from about 2:1 to about 100:1. Typically, the thickness of an elastic strand may be in the range of about 0.02 mm to about 5 mm and the basis weight is in the range of about 20 g/m² to about 300 g/m^{2.}

Any topsheet compatible with the present invention which is known in the art can be used in the present invention. A suitable material for a topsheet may be manufactured from a wide range of materials, such as porous foams, reticulated foams, apertured plastic films, or woven or nonwoven materials of natural fibers (e.g., wood or cotton fibers), synthetic fibers (e.g., polyester or polypropylene fibers), or a combination of natural and synthetic fibers. As an example, a material suitable for use in a topsheet comprises a web of staple-length polypropylene fibers is manufactured by Veratec, Inc., a Division of International Paper Company, of Walpole, Mass. under the designation P-8.

Some examples of suitable topsheets are described further in U.S. Pat. No. 3,929,135; U.S. Pat. No. 4,324,246; U.S. Pat. No. 4,342,314; U.S. Pat. No. 4,463,045; U.S. Pat. No. 5,006,394; U.S. Pat. No. 4,609,518; U.S. Pat. No. 4,629,643. Any portion of the topsheet may be coated with a lotion as is known in the art. Examples of suitable lotions include those described in U.S. Pat. No. 5,607,760; U.S. Pat. No. 5,609,587; U.S. Pat. No. 5,635,191; U.S. Pat. No. 5,643,588; U.S. Pat. No. 5,968,025; U.S. Pat. No. 6,716,441; and PCT Publication No. WO 95/24173.

Further, the topsheet may be fully or partially elastically extensible or may be foreshortened so as to provide a void space between the topsheet and the absorbent core. Exemplary structures including elasticized or foreshortened topsheets are described in more detail in U.S. Pat. No. 4,892,536; U.S. Pat. No. 4,990,147; U.S. Pat. No. 5,037,416; and U.S. Pat. No. 5,269,775.

A suitable backsheet for use in the disposable absorbent article of the present invention may comprise a laminated structure. For example, as previously discussed, the backsheet may comprise a first backsheet layer and a second backsheet layer (see items 241 and 242 of FIG. 2C). The second backsheet layer can be impervious to liquids (e.g., urine) and comprise a thin plastic film such as a thermoplastic film having a thickness, for example, of about 0.012 mm (0.5 mil) to about 0.051 mm (2.0 mils). Suitable backsheet films include those manufactured by Tredegar Corporation, based in Richmond, Va., and sold under the trade name CPC2 film. Either the first backsheet layer and/or the second backsheet layer may include breathable materials which permit vapors to escape from the pull-on garment while still preventing exudates from passing through the backsheet. Suitable breathable materials may include materials such as woven webs, nonwoven webs, composite materials such as film-coated nonwoven webs, microporous films such as manufactured by Mitsui Toatsu Co., of Japan under the designation ESPOIR NO™ and by Tredegar Corporation of Richmond, Va. and sold under the designation EXAIRE™, and monolithic films such as manufactured by Clopay Corporation, Cincinnati, Ohio under the name HYTREL blend P18-3097. Some breathable composite materials are described in greater detail in PCT Application No. WO 95/16746; U.S. Pat. No. 5,938,648; U.S. Pat. No. 5,865,823; and U.S. Pat. No. 5,571,096.

The backsheet, or any portion thereof, may be elastically extensible in one or more directions. In one embodiment, the backsheet may comprise a structural elastic-like film (“SELF”) web. A structural elastic-like film web is an extensible material that exhibits an elastic-like behavior in the direction of elongation without the use of added elastic materials and is described in more detail in U.S. Pat. No. 5,518,801. In alternate embodiments, the backsheet may comprise elastic films, foams, strands, or combinations of these or other suitable materials with nonwovens or synthetic films.

In some embodiments, the backsheet of the absorbent article includes a substantially vapor permeable material. For example, in some embodiments, the backsheet can be constructed to be permeable to at least water vapor and can have a moisture vapor transmission rate (MVTR) of at least 1000 g/m²/24 hr., preferably at least 1500 g/m²/24 hr., more preferably at least 2000 g/m²/24 hr., and even more preferably at least 3000 g/m²/24 hr. In some embodiments, the backsheet may have a moisture vapor transmission rate of from 1000 to 6000 g/m²/24 hr. or any individual number within the range. Some breathable backsheet materials are described in greater detail in PCT Application No. WO 95/16746; U.S. Pat. No. 5,938,648; U.S. Pat. No. 5,865,823; and U.S. Pat. No. 5,571,096. Other suitable exemplary materials and a suitable test method for measuring the MVTR is described in U.S. Pat. No. 6,448,467.

A suitable absorbent core for use in the present invention may comprise any absorbent material which is generally compressible, conformable, non-irritating to the wearer's skin, and capable of absorbing and retaining liquids such as urine and other certain body exudates. In addition, the configuration and construction of the absorbent core may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). Suitable exemplary absorbent structures for use as the absorbent core are described in U.S. Pat. No. 4,610,678; U.S. Pat. No. 4,673,402; U.S. Pat. No. 4,834,735; U.S. Pat. No. 4,888,231; U.S. Pat. No. 5,137,537; U.S. Pat. No. 5,147,345; U.S. Pat. No. 5,342,338; U.S. Pat. No. 5,260,345; U.S. Pat. No. 5,387,207; and U.S. Pat. No. 5,625,222.

The backsheet may be attached to the topsheet, the absorbent core, or any other element of the disposable absorbent article by any attachment means known in the art. For example, the attachment means may include a uniform continuous layer of adhesive, a patterned layer of adhesive, or an array of separate lines, spirals, or spots of adhesive. Some suitable attachment means are disclosed in U.S. Pat. No. 4,573,986; U.S. Pat. No. 3,911,173; U.S. Pat. No. 4,785,996; and U.S. Pat. No. 4,842,666. Examples of suitable adhesives are manufactured by H. B. Fuller Company of St. Paul, Minn. and marketed as HL-1620 and HL-1358-XZP. Alternatively, the attachment means may comprise heat bonds, pressure bonds, ultrasonic bonds, dynamic mechanical bonds, or any other suitable attachment means or combinations of these attachment means as are known in the art.

Various sublayers may be disposed between the topsheet and the backsheet. The sublayer may be any material or structure capable of accepting, storing or immobilizing bodily exudates. Thus, the sublayer may include a single material or a number of materials operatively associated with each other. Further, the sublayer may be integral with another element of the pull-on disposable absorbent article or may be one or more separate elements attached directly or indirectly with one or more elements of the disposable absorbent article. Further, the sublayer may include a structure that is separate from the absorbent core or may include or be part of at least a portion of the absorbent core.

Suitable exemplary materials for use as the sublayer may include large cell open foams, macro-porous compression resistant nonwoven highlofts, large size particulate forms of open and closed cell foams (macro and/or microporous), highloft nonwovens, polyolefin, polystyrene, polyurethane foams or particles, structures comprising a multiplicity of vertically oriented looped strands of fibers, absorbent core structures described above having punched holes or depressions, and the like. (As used herein, the term “microporous” refers to materials which are capable of transporting fluids by capillary action. The term “macroporous” refers to materials having pores too large to effect capillary transport of fluid, generally having pores greater than about 0.5 mm in diameter and, more specifically, having pores greater than about 1.0 mm in diameter.) One embodiment of a sublayer includes a mechanical fastening loop landing element, having an uncompressed thickness of about 1.5 millimeters available as XPL-7124 from the 3M Corporation of Minneapolis, Minn. Another embodiment includes a 6 denier, crimped and resin-bonded nonwoven highloft having a basis weight of 110 grams per square meter and an uncompressed thickness of 7.9 millimeters which is available from the Glit Company of Wrens, Ga. Other suitable absorbent and nonabsorbent sublayers are described in U.S. Pat. No. 6,680,422 and U.S. Pat. No. 5,941,864. Further, the sublayer, or any portion thereof, may include or be coated with a lotion or other known substances to add, enhance or change the performance or other characteristics of the element.

Embodiments of the present invention may also include pockets for receiving and containing waste, spacers which provide voids for waste, barriers for limiting the movement of waste in the article, compartments or voids which accept and contain waste materials deposited in the pull-on disposable absorbent article, and the like, or any combinations thereof. Examples of pockets and spacers for use in absorbent products are described in U.S. Pat. No. 5,514,121; U.S. Pat. No. 5,171,236; U.S. Pat. No. 5,397,318; U.S. Pat. No. 5,540,671; U.S. Pat. No. 6,168,584; U.S. Pat. No. 5,306,266; and U.S. Pat. No. 5,997,520. Examples of compartments or voids in an absorbent article are disclosed in U.S. Pat. No. 4,968,312; U.S. Pat. No. 4,990,147; U.S. Pat. No. 5,062,840; and U.S. Pat. No. 5,269,755. Examples of suitable transverse barriers are described in U.S. Pat. No. 5,554,142; PCT Patent WO 94/14395; and U.S. Pat. No. 5,653,703. Examples of other structures suitable for management of low viscosity feces are disclosed in U.S. Pat. No. 5,941,864; U.S. Pat. No. 5,977,430; and U.S. Pat. No. 6,013,063.

Embodiments of the present invention may include acquisition/distribution layers which can be configured to distribute moisture from a wetness event to moisture responsive members within the disposable absorbent article. Examples of suitable acquisition/distribution layers are described in U.S. Pat. No. 5,460,622, U.S. Patent Application Publication No. 2005/0027267, and U.S. Patent Application Publication No. 2005/009173.

Embodiments of the present invention may include a dusting layer which is well known in the art. Examples of suitable dusting layers are discussed in U.S. Pat. No. 4,888,231.

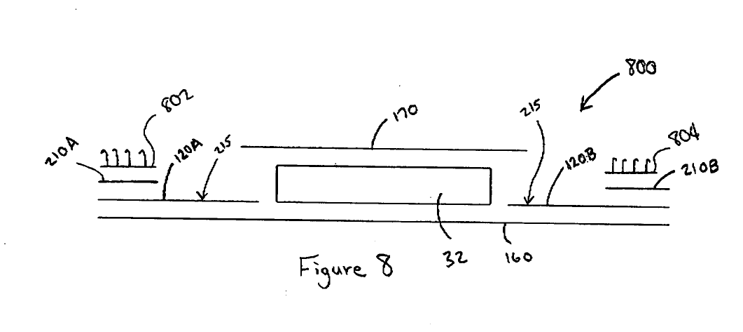

A disposable absorbent article constructed in accordance with the present invention may comprise fastening elements. As shown in FIG. 8, in some embodiments, a disposable absorbent article 800 constructed in accordance with the present invention may comprise fastening elements which are capable of maintaining the front waist region and the back waist region in a configuration so as to provide lateral tensions about the circumference of the disposable absorbent article to hold the disposable absorbent article on the wearer. As shown, in some embodiments, engagement components 802 and 804 can be joined to the wearer-facing surface 215 of the first side panel 120A and the first side panel 120B. In some embodiments, the reinforcement elements 210A and 210B can be disposed between the engagement components 802 and 804 and the first side panels 120A and 120B, respectively. However, the reinforcement elements 210A and 210B can be configured in any suitable manner as discussed herein with regard to the engagement components 802 and 804.

The engagement components 802 and 804 can be configured to join to corresponding receiving components (not shown) in the second waist region of the disposable absorbent article 800, thereby creating a waist opening and a pair of leg openings.

The fastening elements preferably comprise a surface fastener such as tape tabs, hook and loop fastening components, hook and hook, and/or hermaphroditic fastening components, although any other known fastening means are generally acceptable. In alternative embodiments, opposing sides of the article may be seamed or welded to form a pant. This can allow the article to be used as a pull-on type diaper, such as a training pant.

Some exemplary surface fastening systems are disclosed in U.S. Pat. No. 3,848,594, U.S. Pat. No. 4,662,875, U.S. Pat. No. 4,846,815, U.S. Pat. No. 4,894,060, U.S. Pat. No. 4,946,527, U.S. Pat. No. 5,151,092, and U.S. Pat. No. 5,221,274. An exemplary interlocking fastening system is disclosed in U.S. Pat. No. 6,432,098. The fastening system may also: provide a means for holding the article in a disposal configuration as disclosed in U.S. Pat. No. 4,963,140; include primary and secondary fastening systems, as disclosed in U.S. Pat. No. 4,699,622; provide means to reduce shifting of overlapped portions or to improve fit as disclosed in U.S. Pat. No. 5,242,436; and provide means to resist gapping at a wearer's belly as disclosed in U.S. Pat. No. 5,499,978, U.S. Pat. No. 5,507,736, and in U.S. Pat. No. 5,591,152.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

What is claimed is:

1. A disposable absorbent article for wearing about the lower torso of a wearer, the disposable absorbent article comprising: a first waist region, a second waist region, a crotch region disposed between the first waist region and the second waist region; a first waist edge and a second waist edge; and a first longitudinal edge and a second longitudinal edge; the disposable absorbent article further comprising:
a chassis comprising:
a topsheet;
a backsheet attached to at least a portion of the topsheet; and
an absorbent core disposed between the topsheet and the backsheet;
a first side panel extending outward from the first longitudinal edge in the first waist region, wherein the first side panel comprises an inner end and an outer end;
a second side panel extending outward from the first longitudinal edge in the second waist region, wherein the second side panel is configured to join to the first side panel adjacent to the outer end of the first side panel; and
a first reinforcement element joined to the outer end of the first side panel, wherein the first reinforcement element is limited to the outer end of the first side panel.

2. The disposable absorbent article of claim 1, wherein the first side panel and the second side panel are elastically extensible.

3. The disposable absorbent article of claim 1, wherein the reinforcement element comprises a portion of the backsheet.

4. The disposable absorbent article of claim 3, wherein the reinforcement element is disposed on a wearer-facing surface of the disposable absorbent article.

5. The disposable absorbent article of claim 3, wherein the reinforcement element is disposed on an outer-facing surface of the disposable absorbent article.

6. The disposable absorbent article of claim 3, wherein the reinforcement element is disposed between the first side panel and the backsheet.

7. The disposable absorbent article of claim 1, wherein the first side panel comprises an elastomeric element joined to a cover layer such that the elastomeric element and the cover layer are in a face to face orientation.

8. The disposable absorbent article of claim 7, wherein the reinforcement element comprises a portion of the cover layer.

9. The disposable absorbent article of claim 8, wherein the reinforcement element is disposed on an outer-facing surface of the disposable absorbent article.

10. The disposable absorbent article of claim 8, wherein the reinforcement element is disposed between the cover layer and the elastomeric element.

11. The disposable absorbent article of claim 8, wherein the reinforcement element is disposed between the elastomeric element and the backsheet.

12. The disposable absorbent article of claim 1, wherein the second side panel further comprises a second inner end, a second outer end, and a second reinforcement element, wherein the second reinforcement element is limited to the second outer end of the second side panel, wherein the second side panel is joined to the first side panel adjacent the second outer end, and wherein the second reinforcement element overlaps a portion of the first side panel and overlaps a portion of the second side panel.

13. The disposable absorbent article of claim 7, wherein the elastomeric element and the reinforcement element are in a non-overlapping configuration.

14. A disposable pant-like absorbent article for wearing about the lower torso of a wearer, the disposable pant-like absorbent article including an outer cover having a front waist region, a back waist region, and a crotch region disposed between the front waist region and the back waist region, a first backsheet layer which defines an outer surface of the disposable pant-like absorbent article, the disposable pant-like absorbent article further comprising:
a first elastically extensible front side panel and a second elastically extensible front side panel, wherein each of the front side panels are joined to a portion of the first backsheet layer and extend outward from the first backsheet layer, wherein the first and the second the front side panels comprise a first inner end and a first outer end, wherein first front side panel comprises a first reinforcement element joined to the first outer end and limited to the first outer end of the first front side panel, and wherein the second front side panel comprises a first reinforcement element joined to the first outer end and limited to the first outer end of the second front side panel;
a first elastically extensible back side panel and a second elastically extensible back side panel, wherein each of the back side panels are joined to a portion of the first backsheet layer and extend outward from the first backsheet layer, wherein the first and the second back side panels comprise a second inner end and a second outer end, wherein the first back side panel comprises a second reinforcement element joined to the second outer end and limited to the second outer end of the first back side panel, and wherein the second back side panel comprises a second reinforcement element joined to the second outer end and limited to the second outer end of the second back side panel;
an absorbent assembly having a first longitudinal edge and a second longitudinal edge, wherein the absorbent assembly comprises a topsheet, a second backsheet layer associated with the topsheet and an absorbent core disposed between said topsheet and the second backsheet layer, wherein the second backsheet layer is disposed on the first backsheet layer, wherein the absorbent assembly is attached to the wearer-facing surface of the disposable pant-like absorbent article such that the first and second longitudinal edges are in a spaced apart relationship with each of the front and back side panels, thereby defining a front longitudinally orientated non-elasticized portions and a rear longitudinally orientated non-elasticized portions therebetween;
a first barrier cuff attached to the first longitudinal edge of the absorbent assembly; and
a second barrier cuff attached to the second longitudinal edge of the absorbent assembly,
wherein the first and the second elastically extensible front side panels are configured to join to the first and the second elastically extensible back side panels thereby creating a waist opening and a pair of leg openings.

15. The disposable absorbent article of claim 14, wherein the first reinforcement elements and the second reinforcement elements are disposed on a wearer-facing surface of the disposable absorbent article.

16. The disposable absorbent article of claim 14, wherein the first reinforcement elements and the second reinforcement elements are disposed on an outer-facing surface of the disposable absorbent article.

17. The disposable absorbent article of claim 14, wherein the first reinforcement elements are disposed between the first backsheet layer and the first front side panel and the second front side panel and the second reinforcement elements are disposed between the first backsheet layer and the first back side panel and the second back side panel.

18. The disposable absorbent article of claim 14, wherein each of the first and the second front side panels and each of the first and the second back side panels comprise an elastomeric element joined to a cover layer such that the elastomeric element and the cover layer are in a face to face orientation.

19. The disposable absorbent article of claim 18, wherein the first reinforcement elements and the second reinforcement elements are disposed between the cover layer and the elastomeric element.

20. The disposable absorbent article of claim 18, wherein the first reinforcement element and the elastomeric elements are configured in a non-overlapping manner, and wherein the second reinforcement elements and the elastomeric elements are configured in a non-overlapping manner.
